(19) United States
(12) Patent Application Publication
(10) Pub. No.: US 2023/0056414 A1
(43) Pub. Date: Feb. 23, 2023

(54) 3D CELL CULTURE GEL KIT AND 3D CELL CULTURE METHOD USING THE SAME

(71) Applicant: GECOLL BIOMEDICAL CO., LTD., TAINAN CITPY (TW)

(72) Inventors: YU-CHUN WU, TAINAN CITY (TW); CHING-WEN LIU, TAINAN CITY (TW); WEI-YU LIN, TAINAN CITY (TW)

(21) Appl. No.: 17/759,124

(22) PCT Filed: Aug. 26, 2020
(86) PCT No.: PCT/CN2020/111261

§ 371 Date: Jul. 20, 2022

(51) Int. Cl. C12N 5/00

(52) U.S. Cl. CPC C12N 5/0062; C12N 2513/00; C12N 2533/54; C12N 2533/74

(57) ABSTRACT

A 3D cell culture gel kit and a 3D cell culture method using the same are provided. The 3D cell culture gel kit includes a gel material A, a buffer solution C, and a buffer solution D. The 3D cell culture method includes the steps of adding cells into a mixed solution containing the gel material A and setting the mixed solution at low temperature to get gel containing the cells. Then adding the buffer solution C to the gel for performing crosslinking. Next removing the buffer solution C and adding a growth medium. Let stand until the cells form spheroids in the gel. Moreover, the buffer solution D is used to dissolve the gel and the cells cultured are taken out for analysis. Thereby the 3D cell culture gel kit is convenient to use and suitable for 3D culture of a plurality of cell lines.

### BACKGROUND OF THE INVENTION

### Field of the Invention

The present invention relates to a 3D cell culture gel kit and a 3D cell culture method using the same, especially to a 3D cell culture gel kit and a 3D cell culture method using the same which are convenient to use and suitable for 3D cell culture of a plurality of cell lines.

### Description of Related Art

Since in vitro cell culture technique has been quite mature, cell lines are often used in biological research now. Most of the methods of growing cell line are two-dimensional (2D) cell cultures which grow cells on a flat surface such as the bottom of a Petri dish. However, most of cells in organisms grow in a three-dimensional (3D) environment. Thus there is a considerable difference between the 2D cell culture and in vivo cell culture. In order to imitate growth in the organisms, a 3D cell culture technique which grow cells in 3D culture matrix used in biological research has been developed. Refer to Chinese Pat. No. CN 105695413(B), a three-dimensional (3D) cell culture material for individualized tumor therapies and a method of preparing the same are revealed. A plurality of materials including Matrigel, low-viscosity sodium alginate solution, transparent sodium hyaluronate (SH), and medium-viscosity sodium alginate solution is used as 3D cell culture matrix. However, most of the matrix used in the 3D cell cultures now is Matrigel which has complicated preparation and complex ingredients. The conditions and concentrations during which the gel is formed are difficult to control. Matrigel will solidity at room temperature due to low gelation temperature and this leads to short operation time so that the operation is easy to fail. Moreover, cost of the 3D culture matrix based on Matrigel is quite high. Thus 3D cell culture available now is limited.

### SUMMARY OF THE INVENTION

The materials and the method for 3D cell culture available now still is have certain shortcomings while in use. Thus there is a room for improvement and there is a need to provide a novel 3D cell culture material and a 3D cell culture method using the same.

Therefore, it is a primary object of the present invention to provide a 3D cell culture gel kit and a 3D cell culture method using the same. The 3D cell culture gel kit includes a gel material A, a buffer solution C, and a buffer solution D. The gel material A consists of 0.5-3 wt % sodium alginate, 2.5-15 wt % gelatin, 0.5-2 wt % HEPES (4-(2-hydroxyethyl) piperazineethanesulfonic acid) buffer solution, and the remaining percentage of pure water. The buffer solution C contains 0.2-10 wt % salts of divalent cations, 0.01-1 wt % HEPES buffer solution, and the remaining percentage of pure water. The buffer solution D contains 1-10 wt % disodium ethylenediaminetetraacetate (disodium ethylenediaminetetraacetate.2H₂O), 0.1-1 wt % sodium hydroxide (NaOH), 0.05-1 wt % HEPES buffer solution, and the remaining percentage of pure water.

The 3D cell culture method using the 3D cell culture gel kit according to the present invention includes the following steps. (a) Suspending cells in a growth medium to get a cell suspension and then mixing the cell suspension with gel material A in a volume ratio of 1:1 to obtain a mixed solution; (b) arranging a Petri dish at a cryogenic component, adding the mixed solution into wells of the Petri dish, and allowing the mixed solution to stand for 1-7 minutes to form gel; (c) adding buffer solution C to the gel and standing for 10-20 minutes to carry out crosslinking; (d) replacing the buffer solution C with a growth medium for the cells; and (e) arranging the Petri dish at a temperature required until the cells form spheroids in the gel while the growth medium in the Petri dish needs to be replaced once a day.

Preferably, the salts of the divalent cations of the buffer solution C can be strontium chloride, calcium phosphate, calcium chloride, calcium sulfate, or their combinations.

Preferably, the HEPES buffer solution is 0.5-3 M HEPES buffer solution.

Preferably, the 3D cell culture gel kit further includes a thermal conductive sheet.

Preferably, a step (f) is run after the step (e). The step (f) is washing the spheroids with phosphate buffer solution (1× PBS), removing the phosphate buffer solution and then adding buffer solution D and mixing the buffer solution D with the spheroids evenly. Next reacting at room temperature for 5 minutes to make the gel dissolve while the buffer is solution D contains 1-10 wt % disodium ethylenediaminetetraacetate (disodium ethylenediaminetetraacetate.2H₂O), 0.1-1 wt % sodium hydroxide (NaOH), 0.05-1 wt % HEPES buffer solution, and the remaining percentage of pure water.

Preferably, a step (g) is carried out after the step (f). The step (g) is washing dissolved gel solution with phosphate buffer solution and collecting cell precipitate for analysis of cells in the cell precipitate after centrifugation and removal of supernatant.

Preferably, the cryogenic component mentioned in the step (b) is a thermal conductive sheet which has been cooled on a cryogenic device.

Preferably, temperature required for the cell culture is 30-37° C. .

Preferably, cell density of the mixed solution is 10⁴-10⁷ cells/mL.

Therefore, the 3D cell culture gel kit of the present invention is convenient to use and suitable for 3D culture of a plurality of cell lines. Moreover, the present kit also includes buffer solution D able to dissolve the gel with cells. Thus subsequent cell analysis after the culture is also quite convenient.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a schematic drawing showing a flow chart of a 3D cell culture method of an embodiment according to the present invention;

FIG. 2 is a schematic drawing showing a flow chart of a method of dissolving a gel for cell culture to analyze cells of an embodiment according to the present invention;

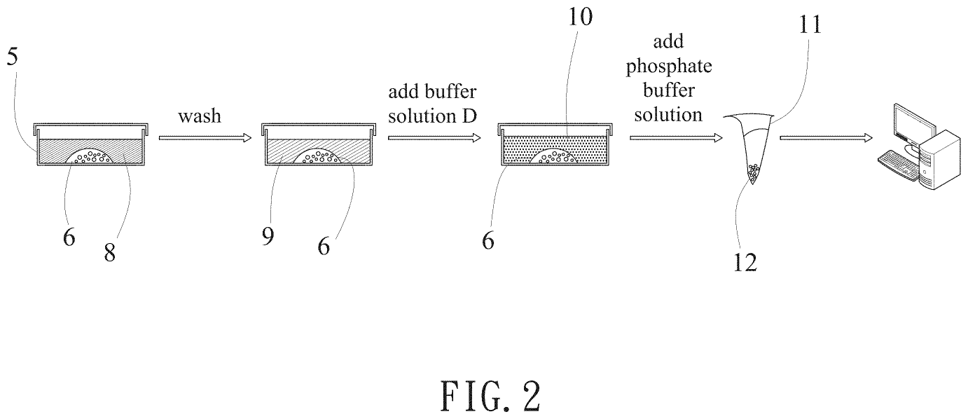

FIG. 3 is a result of cell viability test after cell culture of an embodiment according to the present invention;

FIG. 4A is a microscopic image of 3D cell culture with Matrigel;

FIG. 4B is a microscopic image of 3D cell culture with an embodiment according to the present invention;

FIG. 5 are microscopic images showing cell aggregates formed after cell cultures with an embodiment according to the present invention.

### DETAILED DESCRIPTION OF THE PREFERRED EMBODIMENT

In order to learn technical content, purposes and functions of the present invention more clearly and completely, please refer to the is following detailed descriptions with the figures and reference signs.

A three-dimensional (3D) cell culture gel kit and a 3D cell culture method using the same are provided. The kit is convenient to use and suitable for 3D cell culture of a plurality of cell lines. Moreover, the kit also includes a buffer solution able to dissolve the gel and making subsequent cell analysis more convenient.

The 3D cell culture gel kit according to the present invention mainly includes a gel material A, a buffer solution C, and a buffer solution D. The present kit can further include a cryogenic component (such as thermal conductive sheet) for assistance in formation of the gel during 3D cell culture.

The gel material A of the 3D cell culture gel kit consists of sodium alginate, gelatin, HEPES (4-(2-hydroxyethyl)-1-piperazineethanesulfonic acid) buffer solution, and the remaining percentage of pure water.

Moreover, the buffer solution C contains 0.2-10 wt % salts of divalent cations such as strontium chloride (Strontium Chloride.6H₂O), calcium phosphate, calcium chloride, and calcium sulfate, 0.01-1 wt % HEPES buffer solution, and the remaining percentage of pure water. In a preferred embodiment, the buffer solution C includes 0.5 wt % strontium chloride, 0.094 wt % 1 M HEPES buffer solution, and the remaining percentage of pure water. For ease of use, the buffer solution C can be prepared in the form of a concentrated buffer solution C such as 10× concentrated buffer solution C which contains a high concentration of salts. The concentrated buffer solution C is diluted before use. In the following embodiments, the buffer solution C is added with strontium chloride as the salt of divalent cation.

As to the buffer solution D, it contains 1-10 wt % disodium ethylenediaminetetraacetate (disodium ethylenediaminetetraacetate.2H₂O), 0.1-1 wt % sodium hydroxide (NaOH), 0.05-1 wt % HEPES buffer solution, and the remaining percentage of pure water. In a preferred embodiment, the buffer solution D is composed of 1.53 wt % disodium ethylenediaminetetraacetate (disodium ethylenediaminetetraacetate.2H₂O), 0.164 wt % sodium hydroxide (NaOH), 0.082 wt % 1M HEPES buffer solution, and the remaining percentage of pure water. For ease of use, the buffer solution D solution can be prepared in the form of a highly-concentrated buffer solution D such as 10× concentrated buffer solution D which contains a high concentration of salts. The concentrated buffer solution D is diluted before use.

Before cell culture, all the material needed are prepared. The gel material A is placed in a 37° C. cell bath for about 10 minutes to be dissolved completely. Then the 10× buffer solution C is diluted into 1× buffer solution C with a serum-free cell culture medium. It should be noted that 1× PBS buffer solution can't be used to dilute the 10× buffer solution C in this step. Next the 10× buffer solution D is diluted with the 1× PBS buffer to get 1× buffer solution D. It should be noted that both the 1× buffer solution C and the 1× buffer solution D are diluted only before use.

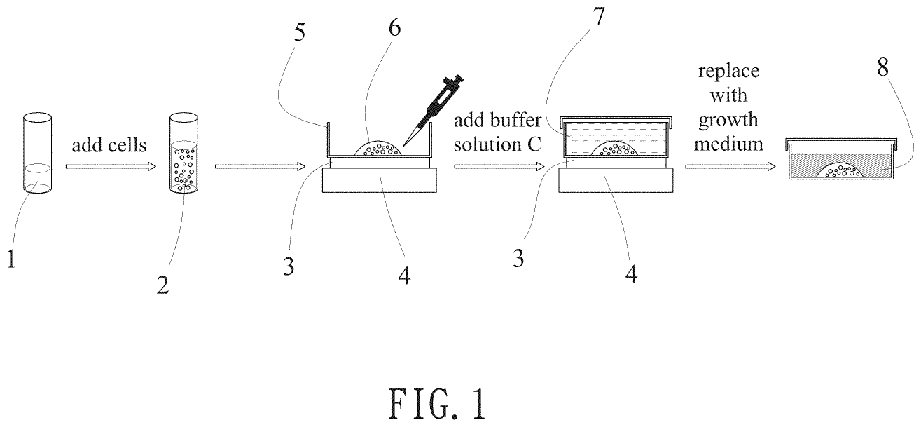

Refer to FIG. 1, a 3D cell culture method using the above 3D cell culture gel kit according to the present invention includes the following steps.

Step (a): first putting 0.5 mL gel material A 1 into a test tube and suspending cells going to be cultured in a growth medium to get a cell suspension; then adding 0.5 mL cell suspension into the test tube with 0.5 mL gel material A 1, mixing the cell suspension and the gel material A 1 well to obtain a mixed solution 2. A cell density in the mixed solution 2 is adjustable depending on the cells being cultured. For example, cells of an adherent cell line being cultured can cover 80% of a surface of a Petri dish. The most common cell density used is 10⁴-10⁷ cells/mL. In this embodiment, the cell density in the mixed solution 2 is 10⁵ cells/mL;

Step (b): arranging a thermal conductive sheet 3 at a cryogenic device 4 so that a temperature of the thermal conductive sheet 3 is decreased quickly and evenly. The thermal conductive sheet 3 can be, but not limited to a metal sheet while the cryogenic device 4 can be, but not limited to an object such as an ice pack or a reusable ice pad. Then a Petri dish 5 is disposed on the thermal conductive sheet 3 already cooled down. Next 20-50 μL mixed solution 2 is added into wells of the Petri dish 5 and the mixed solution 2 is allowed to stand for 1-7 minutes to form gel 6. In order to check the formation of the gel 6, use a pipet tip to touch a surface of the gel 6 gently. Once the gel 6 is formed, the surface of the gel 6 will not be pulled outward when the pipet tip is moved away from the surface of the gel 6;

Step (c): adding chilled 1 mL 1× buffer solution C 7 at a temperature of about 4° C. to the gel 6. Then the buffer solution C 7 covers the gel 6 and let stand for 10-20 minutes to carry out crosslinking In this embodiment, allow the gel 6 with the buffer solution C 7 to stand for 15 minutes;

Step (d): removing the 1× buffer solution C 7 carefully and then adding the growth medium 8 for the cells being cultured;

Step (e): disposing the Petri dish 5 at a place with temperature and conditions required and replacing the growth medium 8 once a day until the cells form spheroids in the gel 6. The temperature required in the step (e) means preferred temperature at which the cells are cultured. For instance, 37° C. is the most commonly used for mammal cells. As to culture time, it depends on cell types. Generally, the spheroids formed by the cells in the gel 6 are observed after the cells being cultured for 3-14 days.

After growth of the cells, step (f) and step (g) are executed after the step (e) to take the cells in the gel 6 out and perform the following observations and analysis.

Step (f): Removing the growth medium 8 in the Petri dish 5, washing the gel 6 with chilled phosphate buffer solution 9 such as 1× PBS having a temperature of about 4° C., and then removing the phosphate buffer solution 9. Run the washing step at least once. Next add the chilled buffer solution D 10 at a temperature of about 4° C. into the gel 6 and mix the buffer solution D 10 with the gel 6 evenly. React at room temperature for about 5 minutes to make the gel 6 dissolve.

Step (g): Taking a mixed solution of the dissolved gel 6 and the cells out of the Petri dish 5, putting the mixed solution into a centrifuge tube 11, and adding the phosphate buffer solution 9 into the centrifuge tube 11 for washing the mixed solution of the dissolved gel 6 and the cells. Then carry out centrifugation at the speed without damaging the cells such as 1000rpm for 10 minutes and collect cell precipitate 12 after is removal of supernatant.

In order to perform further analysis of a single kind of cells, the cell precipitate 12 is treated by a trypsin solution (trypsin-EDTA solution) for separation of the cells in the spheroids to get a single cell.

### Embodiment 1: Gel Formation Test of Gel Material A

Please refer to the following Table 1, the inventor of the present kit and method has tested an impact of a ratio of sodium alginate, gelatin, and HEPES buffer solution in the gel material A on gel formation. Seven formulas are provided in the Table 1 and gel formed by the respective formulas has been tested. The gel can be produced by each of the seven formulas of the gel material A. “Gel sliding” means whether the gel is sliding with respect to the bottom of the Petri dish. “Light transmittance” means whether words on a paper can be observed through the gel placed on the paper with the words. “No disruption time” means a period during which morphology of the gel is maintained. According to the Table 1, the gels produced by the seven formulas all have good light transmittance and the no disruption time is longer than at least 12 days. Thus the gels are really suitable for 3D cell culture. Moreover, the gel 6 formed by the formula 6 of the gel material A in the Table 1 is used to carry out 3D cell culture in the following embodiments.

TABLE 1

| | sodium | | | | | | no |
|---|---|---|---|---|---|---|---|
| | alginate | gelatin | HEPES | gel | gel | light | disruption |
| | (wt %) | (wt %) | (M) | formation | sliding | transmittance | time (day) |
| formula 1 | 1.5 | 10 | 0.01 | capable | no | transparent | >14 |
| formula 2 | 1 | 4 | 0.01 | capable | yes | transparent | >12 |
| formula 3 | 2 | 8 | 0.01 | capable | no | transparent | >14 |
| formula 4 | 1.5 | 6 | 0.01 | capable | no | transparent | >14 |
| formula 5 | 1 | 6 | 0.01 | capable | no | transparent | >12 |
| formula 6 | 1.5 | 8 | 0.01 | capable | no | transparent | >14 |
| formula 7 | 3 | 12 | 0.01 | capable | no | transparent | >28 |

### Embodiment 2: Cell Viability Test

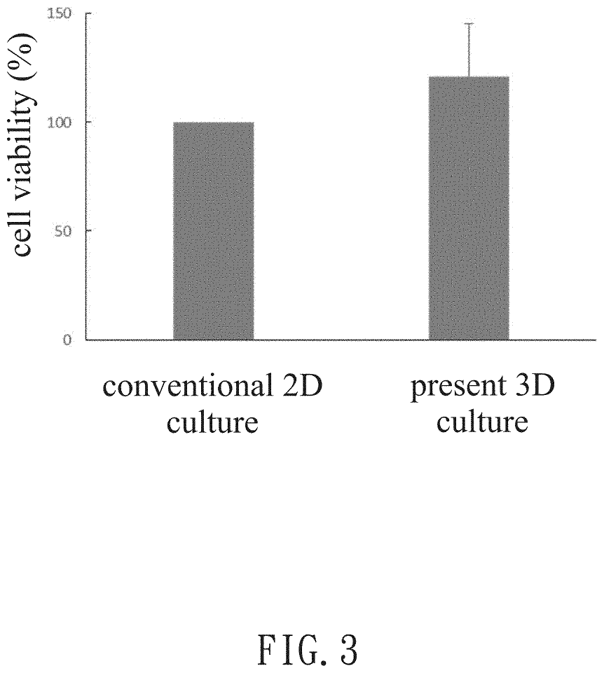

Huh-7 cells are cultured by the 2D method or the 3D method with the kit of the present invention. After being cultured for 48 hours, assess cell viability by performing MTT (3-(4,5-Dimethylthiazol-2-yl)-2,5-diphenyltetrazolium bromide assay. Since the MTT assay is a common test for evaluation of cell survival in the field, the related steps are not explained here. As shown in FIG. 3, the number of the live Huh-7 cells after being cultured by the kit is significantly increased compared with that at the start of the culture. This means the kit of the present invention has no cytotoxicity, indeed able to be used in cell culture.

### Embodiment 3: Growth Test of Spheroids

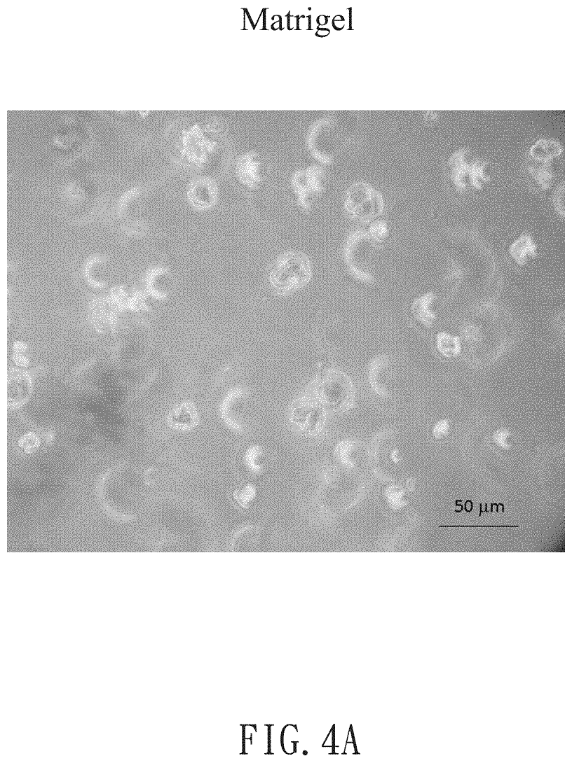

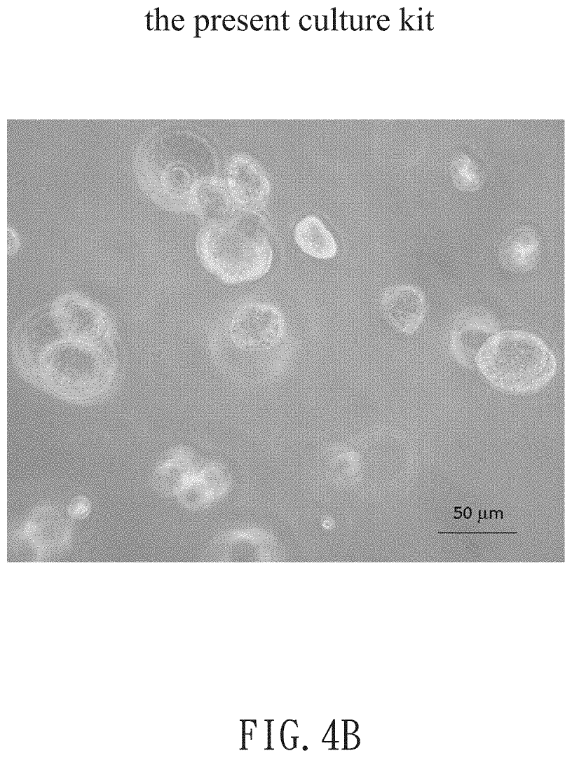

In this embodiment, culture 1×10⁵ Huh-7 cells in 3D culture is initially with Matrigel and the present kit and then observe the growth of spheroids in the two groups of Huh-7 cells. Refer to FIGS. 4A and 4B, no spheroids are observed at the 4th day of culture in the Matrigel-cultured cells while spheroids with a diameter of about 20 mm are formed in the group cultured in the present kit. At the 7^{th} day of culture, the diameters of the spheroids in the groups cultured in the Matrigel and the present kit are about 50 mm and 100 mm respectively. Obviously the spheroids are formed earlier and grown better in the group cultured in the present kit compared with the group cultured in Matrigel.

### Embodiment 4: Culturable Cell Types

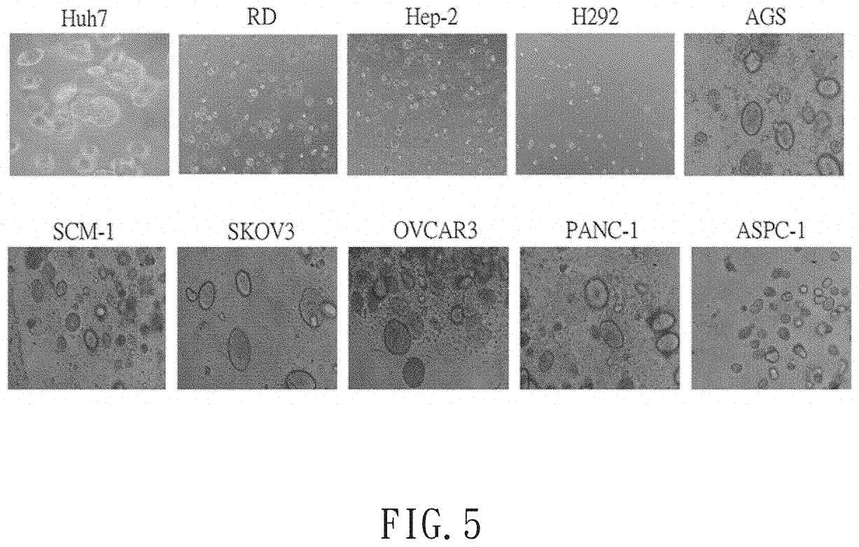

Refer to Table 2 and FIG. 5, a plurality of cancers cells is cultured in the 3D cell culture gel kit of the present invention and results as well as time required for spheroid formation are shown in the Table 2 and FIG. 5. All of the tested cell lines are observed to grow and form the spheroids no more than 5 days. The results show that the present kit can be applied to a plurality types of cell lines and spheroid formation is observed within a short period of time.

TABLE 2

| | cell density at the | time required for |
|---|---|---|
| cell line | start of the culture | spheroid formation |
| Huh 7 | 1 × 10⁵ cells/mL | 3 |
| RD | 1 × 10⁵ cells/mL | 4 |
| Hep-2 | 1 × 10⁵ cells/mL | 4 |
| H292 | 1 × 10⁵ cells/mL | 4 |
| AGS | 3 × 10⁵ cells/mL | 3 |
| SCM-1 | 3 × 10⁵ cells/mL | 3 |
| SK-OV-3 | 3 × 10⁵ cells/mL | 4 |
| OVCAR3 | 3 × 10⁵ cells/mL | 4 |
| PANC-1 | 3 × 10⁵ cells/mL | 4 |
| AsPC-1 | 3 × 10⁵ cells/mL | 5 |

In summary, a 3D cell culture gel kit and a 3D cell culture method using the same are provided by the present invention. The steps of gel formation are carried out at two stages. First, gel material A is operated in a low-temperature environment such as an environment under 10° C. to form gel. Then the gel reacts with buffer solution C at room temperature to have crosslinking and get 3D culture matrix for 3D cell culture. The two-stage operation can avoid rapid formation of the gel which further affects the preparation of the 3D culture medium during the operation. The 3D cell culture gel kit is easy to use. Time require for gel formation is short and hardness of the gel is easy to control. Moreover, the gel created by the present kit which has network structure of hydrogel is suitable for 3D cultures of most of cell lines and thus a wide range of applications is provided. Furthermore, compared with 3D cell culture by using Matrigel, formation of spheroids is observed earlier in 3D cell culture using the present 3D cell culture gel kit and experimental efficiency is further improved.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A three-dimensional (3D) cell culture gel kit comprising:
a gel material A which contains 0.5-3 wt % sodium alginate, 2.5-15 wt % gelatin, 0.5-2 wt % 4-(2-hydroxyethyl)-1-piperazineethanesulfonic acid (HEPES) buffer solution, and the remaining percentage of pure water;
a buffer solution C which contains 0.2-10 wt % salts of divalent cations, 0.01-1 wt % the HEPES buffer solution, and the remaining percentage of pure water; and
a buffer solution D having 1-10 wt % disodium ethylenediaminetetraacetate, 0.1-1 wt % sodium hydroxide (NaOH), 0.05-1 wt % the HEPES buffer solution, and the remaining percentage of pure water.

2. The 3D cell culture gel kit as claimed in claim 1, wherein the salts of is the divalent cation of the buffer solution C is selected from the group consisting of strontium chloride, calcium phosphate, calcium chloride, calcium sulfate, and combinations thereof.

3. The 3D cell culture gel kit as claimed in claim 1, wherein the HEPES buffer solution is 0.5-3 M HEPES buffer solution.

4. The 3D cell culture gel kit as claimed in claim 1, wherein the 3D cell culture gel kit further includes a thermal conductive sheet.

5. A 3D cell culture method using the 3D cell culture gel kit as claimed in claim 1 comprising the steps of:
(a) suspending cells in a growth medium to get a cell suspension and then mixing the cell suspension with the gel material A in a volume ratio of 1:1 to obtain a mixed solution;
(b) arranging a Petri dish at a cryogenic component, adding the mixed solution into wells of the Petri dish, and allowing the mixed solution to stand for 1-7 minutes to form gel;
(c) adding buffer solution C to the gel and standing for 10-20 minutes to carry out crosslinking;
(d) replacing the buffer solution C with the growth medium; and
(e) arranging the Petri dish at a temperature required until the cells form spheroids in the gel while the growth medium in the Petri dish needs to be replaced once a day.

6. The method as claimed in claim 5, wherein the gel material A contains 0.5-2 wt % sodium alginate, 2.5-15 wt % gelatin, 0.5-2 wt % 4-(2-hydroxyethyl)-1-piperazineethanesulfonic acid (HEPES) buffer solution, and the remaining percentage of pure water; wherein the buffer solution C contains 0.2-10 wt % salts of divalent cations, 0.01-1 wt % HEPES buffer solution, and the remaining percentage of pure water.

7. The method as claimed in claim 5, wherein the method further includes a step (f) run after the step (e); the step (f) is washing the spheroids with phosphate buffer solution, removing the phosphate buffer solution, and then adding buffer solution D and mixing the buffer solution D with the spheroids evenly; next reacting at room temperature for 5 minutes to make the gel dissolve; wherein the buffer solution D contains 1-10 wt % disodium ethylenediaminetetraacetate, 0.1-1 wt % sodium hydroxide, 0.05-1 wt % the HEPES buffer solution, and the remaining percentage of pure water.

8. The method as claimed in claim 7, wherein the method further includes a step (g) run after the step (f); the step (g) is washing dissolved gel solution with the phosphate buffer solution and collecting cell precipitate for analysis of cells in the cell precipitate after centrifugation and removal of supernatant.

9. The method as claimed in claim 5, wherein the cryogenic component in the step (b) is obtained by a thermal conductive sheet placed and cooled on a cryogenic device; wherein temperature required is 30-37 ° C.; wherein cell density of the mixed solution is 10⁴-10⁷ cells/mL.

10. The method as claimed in claim 6, wherein the salts of the divalent cation of the buffer solution C is selected from the group consisting of strontium chloride, calcium phosphate, calcium chloride, calcium sulfate, and combinations thereof.
